(19) United States
(12) Patent Application Publication
(10) Pub. No.: US 2026/0347299 A1
(43) Pub. Date: Nov. 5, 2026

(54) PHOTOCATALYTIC SYNTHESIS OF ALPHA, BETA UNSATURATED CARBONYL COMPOUNDS AND THEIR INTERMEDIATES

(71) Applicants: JSR CORPORATION, Tokyo (JP); NEW IRIDIUM INC., Superior, CO (US)

(72) Inventors: Takuya SUGURI, Tokyo (JP); Youichirou MARUYAMA, Tokyo (JP); Chern-Hooi LIM, Superior, CO (US); Gang QIAN, Superior, CO (US); Yue LIU, Superior, CO (US); Elana COPE, Superior, CO (US)

(73) Assignees: JSR CORPORATION, Tokyo (JP); NEW IRIDIUM INC., Superior, CO (US)

(21) Appl. No.: 18/995,441

(22) PCT Filed: Jul. 18, 2022
(86) PCT No.: PCT/US2022/037417

§ 371 Date: Jan. 16, 2025

(51) Int. Cl. C07C 51/377; B01J 23/30; B01J 31/02; B01J 35/39

(52) U.S. Cl. CPC C07C 51/377; B01J 23/30; B01J 31/0239; B01J 35/39; B01J 2531/66

(57) ABSTRACT

Described herein is a process of producing α,β unsaturated acids and amides and their intermediates via a photocatalytic reaction in the presence of a photocatalyst, oxygen, and optionally a solvent. The photocatalyst contains a tungstic acid or a salt of a tungstic acid. The process is simple, clean, and energy efficient, and provides good conversion, good selectivity, good turnover, and easy purification of end products.

### BACKGROUND OF THE INVENTION

Unsaturated carbonyl compounds, particularly α,β unsaturated acids and amides (methacrylic acid (MAA), methyl methacrylate (MMA), acrylamide (AA), methacrylamide (MA), etc.) are important compounds in the chemical industry, useful both by themselves as well as in combination with other monomers in, for example, the production of polymers for various applications such as adhesives, binders, coatings, paints, polishes, detergents, flocculants, dispersants, sequestrants, etc. However, fossil-derived α,β unsaturated acids and amides contribute to greenhouse emissions, and as fossil resources are becoming increasingly scarce, more expensive, and subject to regulations, there exists a growing need for non-fossil-derived α,β unsaturated acids and amides made from renewable raw materials, which can be replenished naturally or, e.g., via agricultural techniques. Biologically derived volatile fatty acids (e.g., propionic acid, butyric acid, isobutyric acid, isovaleric acid, valeric acid, isocaproic acid, caproic acid, heptanoic acid, octanoic acid, etc.) provide one possible source of such renewable raw materials.

In addition, and regardless of the source of the raw material, there is a need in the art for a simple, clean, energy efficient process for producing α,β unsaturated acids and amides that provides good conversion, good selectivity, and easy purification of end products.

### BRIEF SUMMARY OF THE INVENTION

Described herein is a simple, clean, energy efficient photocatalytic process for producing α,β unsaturated acids and amides and their intermediates that provides good conversion, good selectivity, good turnover, and easy purification of end products.

In one preferred embodiment, a compound of formula (1):

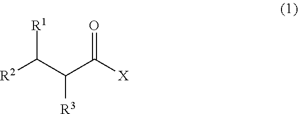

is irradiated with long ultraviolet light and/or short visible light (e.g., 200-500 nm) in the presence of oxygen and a photocatalyst, wherein R¹, R², and R³ each independently represent a hydrogen atom or an organic group having 1-20 carbon atoms, and X represents a monovalent group represented by —OR^{a} or —NR^{b}R^{c}, wherein R^{a}, R^{b}, and R^{c} each independently represent a hydrogen atom or an organic group having 1-20 carbon atoms.

In another embodiment, a compound of formula (3) and/or a compound of formula (3′) are prepared from a compound of formula (1) by oxidizing the compound of formula (1) via a photocatalytic reaction in a presence of oxygen and a photocatalyst:

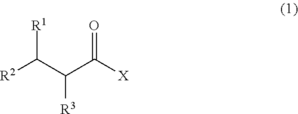

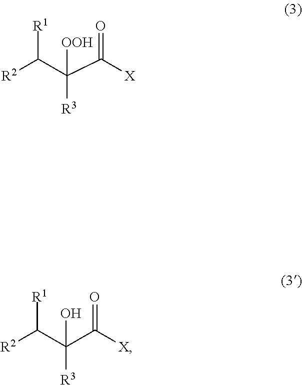

wherein R¹, R², and R³ each independently represent a hydrogen atom or an organic group having 1-20 carbon atoms; and X represents a monovalent group represented by —OR^{a} or —NR^{b}R^{c}, where R^{a}, R^{b}, and R^{c} each independently represent a hydrogen atom or an organic group having 1-20 carbon atoms.

In another preferred embodiment, intermediate compounds of formula (3) and/or formula (3′) are prepared from a compound of formula (1) by oxidizing the compound of formula (1) via a photocatalytic reaction in a presence of oxygen and a photocatalyst:

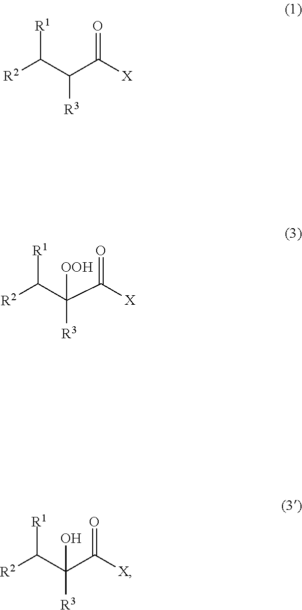

followed by converting the intermediate compounds of formula (3) and/or formula (3′) to a compound of formula (2):

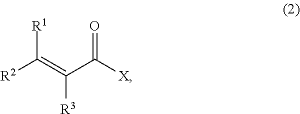

wherein R¹, R², and R³ each independently represent a hydrogen atom or an organic group having 1-20 carbon atoms; and X represents a monovalent group represented by —OR^{a} or —NR^{b}R^{c}, where R^{a}, R^{b}, and R^{c} each independently represent a hydrogen atom or an organic group having 1-20 carbon atoms.

### BRIEF DESCRIPTION OF THE DRAWINGS

FIG. 1A shows conversion of isobutyric acid (IBA) at 0 °C with 2 mM tetrabutylammonium decatungstate (TBADT) photocatalyst in 90% deuterated acetonitrile as a function of time at two different irradiance levels expressed in mW/cm².

FIG. 1B shows conversion of methyl isobutyrate (MIB) at 0 °C with 2 mM TBADT photocatalyst in 90% deuterated acetonitrile as a function of time at two different irradiance levels expressed in mW/cm².

FIG. 2A shows selectivity of desired products versus conversion for IBA at 0 °C with 2 mM TBADT photocatalyst in 90% deuterated acetonitrile at two different irradiance levels expressed in mW/cm².

FIG. 2B shows selectivity of desired products versus conversion for MIB at 0 °C with 2 mM TBADT photocatalyst in 90% deuterated acetonitrile at two different irradiance levels expressed in mW/cm².

FIG. 3A is 1H NMR of the photocatalytic reaction of IBA/TBADT/O₂ in different solvents.

FIG. 3B is 1H NMR of the photocatalytic reaction of MIB/TBADT/O₂ in different solvents.

FIG. 4 is a diagram of an exemplified flow photoreactor setup.

### DETAILED DESCRIPTION OF THE INVENTION

As used herein, where the terms “invention,” “the invention,” “the present invention,” and the like appear in both headings and in text, they refer only to the particular embodiment immediately following. They are not broadly limiting overall, or generally limiting with regard to the several individual advances in the art described herein.

When an amount, concentration, or other value or parameter is given herein as a range, and/or as a list of values, this is to be understood as specifically disclosing all ranges formed from any pair of any upper and lower values, and as including all integers and fractions within the range, regardless of whether all such ranges, integers, and fractions are separately disclosed. Where a range of numerical values is recited herein, unless otherwise stated, the range is intended to include the endpoints thereof, and all integers and fractions within the range. For example, a range of 1-10 includes and discloses 3. It is not intended that the scope of the present invention be limited to the specific values recited when defining a range.

In one embodiment, the present invention provides a method wherein a compound of formula (1):

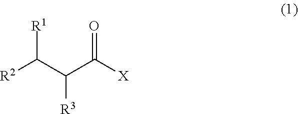

is irradiated with long ultraviolet light and/or short visible light (e.g., 200-500 nm) in the presence of oxygen and a photocatalyst, such as a tungstic acid or a salt thereof, wherein R¹, R², and R³ each independently represent a hydrogen atom or an organic group having 1-20 carbon atoms, and X represents a monovalent group represented by —OR^{a} or —NR^{b}R^{c}, wherein R^{a}, R^{b}, and R^{c} each independently represent a hydrogen atom or an organic group having 1-20 carbon atoms. Preferably, the compound of formula (1) is irradiated for a time and under conditions sufficient to cause it to undergo chemical change (i.e., is transformed into one or more different compounds).

In one embodiment, the present invention provides a method of producing a compound of formula (3), a compound of formula (3′), or both, comprising oxidizing a compound of formula (1) via a photocatalytic reaction in the presence of oxygen and a photocatalyst, such as a tungstic acid or a salt thereof:

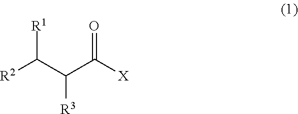

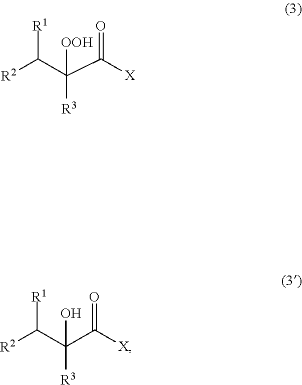

wherein R¹, R², and R³ each independently represent a hydrogen atom or an organic group having 1-20 carbon atoms; and X represents a monovalent group represented by —OR^{a} or —NR^{b}R^{c}, wherein R^{a}, R^{b}, and R^{c} each independently represent a hydrogen atom or an organic group 5 having 1-20 carbon atoms.

In another embodiment, the present invention provides a method of producing a compound of formula (2):

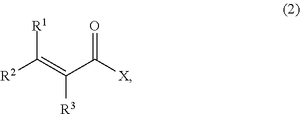

comprising oxidizing a compound of formula (1) via a photocatalytic reaction in the presence of oxygen and a photocatalyst, such as a tungstic acid or a salt thereof, to obtain intermediate compounds of formula (3) and/or formula (3′), and subsequently converting the intermediate compounds of formula (3) and/or formula (3′) to the compound of formula (2):

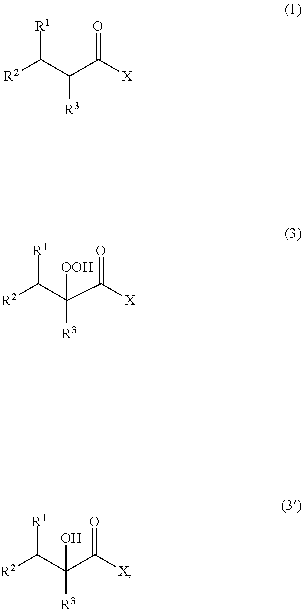

wherein R¹, R², and R³ each independently represent a hydrogen atom or an organic group having 1-20 carbon atoms; and X represents a monovalent group represented by —OR^{a} or —NR^{b}R^{c}, wherein R^{a}, R^{b}, and R^{c} each independently represent a hydrogen atom or an organic group having 1-20 carbon atoms.

Nonlimiting preferred organic groups (organic radicals) having 1-20 carbon atoms for R¹, R², R³, R^{a}, R^{b}, and R^{c} in all formulae and reactions described herein include:
saturated linear or branched-chain hydrocarbon radicals (alkyl radicals). In one embodiment, the alkyl radical is one to eighteen carbon atoms (C₁-C₁₈). In other embodiments, the alkyl radical is C₁-C₆, C₁-C₅, C₁-C₃, C₁-C₁₂, C₁-C₁₀, C₁-C₈, C₁-C₄ or C₁-C₃. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃) (CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃) (CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃) C(CH₃)₃, heptyl, octyl, nonyl, decyl, undecyl and dodecyl. Preferred alkyl groups include methyl, ethyl, 1-propyl, 2-propyl, 1-butyl, 2-methyl-1-propyl, 2-butyl, and 2-methyl-2-propyl;
linear or branched-chain hydrocarbon radicals with at least one carbon-carbon double bond (alkenyl radicals), including those having “cis” and “trans” orientations, or alternatively, “E” and “Z” orientations, all of which are included herein. In one example, the alkenyl radical is two to eighteen carbon atoms (C₂-C₁₈). In other examples, the alkenyl radical is C₂-C₁₂, C₂-C₁₀, C₂-C₈, C₂-C₆ or C₂-C₃. Examples include, but are not limited to, ethenyl or vinyl (—CH═CH), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl;
linear or branched hydrocarbon radicals with at least one carbon-carbon triple bond (alkynyl radicals). In one example, the alkynyl radical is two to eighteen carbon atoms (C₂-C₁₈), In other examples, the alkynyl radical is C₂-C₁₂, C₂-C₁₀, C₂-C₈, C₂-C₆ or C₂-C₃. Examples include, but are not limited to, ethynyl (—C≡CH), prop-1-ynyl (—C≡CCH₃), prop-2-ynyl (propargyl, CH₂C≡CH), but-1-ynyl, but-2-ynyl and but-3-ynyl;
linear or branched radicals represented by the formula —OR in which R is alkyl, alkenyl, alkynyl or carbocycyl (alkoxy groups). Alkoxy groups include methoxy, ethoxy, propoxy, isopropoxy, and cyclopropoxy;
alkyl radicals substituted with one or more (e.g. 1, 2, 3, or 4) halo groups (haloalkyl groups);
alkyl radicals interrupted by one or more (e.g. 1, 2, 3, or 4) oxygen atoms (ether groups);
alkyl radicals interrupted by one or more (e.g. 1, 2, 3, or 4) —COO— or —OCO— groups (ester groups);
monocyclic, bicyclic or tricyclic, carbon ring system groups, that include fused rings. In one embodiment, aryl radicals include groups having 6-20 carbon atoms (C₆-C₂₀ aryl). In another embodiment, aryl includes groups having 6-10 carbon atoms (C₆-C₁₀ aryl). Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, and the like, which may be substituted or independently substituted by one or more substituents described herein;
monocyclic, bicyclic or tricyclic ring system groups having 5 to 14 ring atoms, wherein at least one ring is aromatic and contains at least one heteroatom (heteroaryl radicals) In one embodiment, heteroaryl includes 4-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen that is independently optionally substituted. In some embodiments, the heteroaryl group is a C₁-C₂₀ heteroaryl group, where the heteroaryl ring contains 1-20 carbon atoms and the remaining ring atoms include one or more nitrogen, sulfur, or oxygen atoms. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl, purinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, pyrid-2-yl N-oxide, and pyrazolo[4,3-c]pyridinyl. The term “heteroaryl” also includes groups in which a heteroaryl is fused to one or more aryl, carbocyclyl, or heterocyclyl rings, where the radical or point of attachment is on the heteroaryl ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono-, bi- or tri-cyclic;
saturated, partially unsaturated, or aromatic ring system groups having 3 to 20 carbon atoms (carbocyclyl radicals). In one embodiment, carbocyclyl includes 3 to 12 carbon atoms (C₃-C₁₂). In another embodiment, carbocyclyl includes C₃-C₈, C₃-C₁₀ or C₅-C₁₀. In other embodiment, carbocyclyl, as a monocycle, includes C₃-C₈, C₃-C₆ or C₅-C₆.

In another embodiment, carbocyclyl, as a bicycle, includes C₇-C₁₂. In another embodiment, carbocyclyl, as a spiro system, includes C₅-C₁₂. Examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, for example bicyclo[2.2.1]heptane, bicyclo[2.2.2]octane, naphthalene, and bicyclo[3.2.2]nonane; and spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g. saturated or partially unsaturated mono-, bi-, or spiro-carbocycles). carbocyclyl groups wherein one or more (e.g. 1, 2, 3, or 4) carbon atoms have been replaced with a heteroatom (e.g. O, N, or S) (heterocyclyl or heterocycle radicals). In some embodiments, a heterocyclyl or heterocycle refers to a saturated ring system, such as a 3 to 12 membered saturated heterocyclyl ring system. In some embodiments, a heterocyclyl or heterocycle refers to a heteroaryl ring system, such as a 5 to 14 membered heteroaryl ring system. A heterocyclyl or heterocycle can optionally be substituted with one or more substituents independently selected from those defined herein. In one example, heterocyclyl or heterocycle includes 3-12 ring atoms and includes monocycles, bicycles, tricycles and spiro ring systems, wherein the ring atoms are carbon, and one to five ring atoms is a heteroatom selected from nitrogen, sulfur or oxygen, which is independently optionally substituted by one or more groups. In one example, heterocyclyl or heterocycle includes 1 to 4 heteroatoms. In another example, heterocyclyl or heterocycle includes 3- to 7-membered monocycles having one or more heteroatoms selected from nitrogen, sulfur or oxygen. In another example, heterocyclyl or heterocycle includes 4- to 6-membered monocycles having one or more heteroatoms selected from nitrogen, sulfur or oxygen. In another example, heterocyclyl or heterocycle includes 3-membered monocycles. In another example, heterocyclyl or heterocycle includes 4-membered monocycles. In another example, heterocyclyl or heterocycle includes 5-6 membered monocycles. In one example, the heterocyclyl or heterocycle group includes 0 to 3 double bonds. Any nitrogen or sulfur heteroatom may optionally be oxidized (e.g. NO, SO, SO₂), and any nitrogen heteroatom may optionally be quaternized (e.g. [NR₄]⁺Cl⁻, [NR₄]⁺OH⁻). Example heterocyclyls or heterocycles include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls or heterocycles containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls or heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Example benzo-fused 5-membered heterocyclyls or heterocycles are benzoxazol-2-yl, benzthiazol-2-yl and benzimidazol-2-yl. Example 6-membered heterocyclyls or heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are other example heterocyclyl groups. The term “heterocyclyl” or “heterocycle” also includes groups in which a heterocyclyl is fused to one or more aryl, carbocyclyl, or heterocyclyl rings, where the radical or point of attachment is on the heterocyclyl ring. Nonlimiting examples include tetrahydroquinolinyl and tetrahydroisoquinolinyl. See, e.g., U.S. Pat. No. 11,247,989, incorporated herein by reference.

In preferred embodiments, R¹, R², R³, R^{a}, R^{b}, and R^{e} are each independently a hydrogen atom or an alkyl, alkoxy, haloalkyl, ether, or ester radical. In a preferred embodiment, organic groups having 1-20 carbon atoms preferably have 1-18, 1-16, 1-8, etc. carbons including 1, 2, 3, 4, 5, 6, 7, 8, 9, and 10 carbons.

Preferred compounds of formula (1) include propionic acid, butyric acid, pentanoic acid, hexanoic acid, isobutyric acid, 2-methylbutyric acid, 2-methylpentanoic acid, 2-methylhexanoic acid, the methyl esters of these acids (e.g., methyl propanoate, methyl pentanoate, methyl isobutyrate, etc.), propyl amide, butylamide, 2-methylbutanamide, pentylamide, hexylamide, isobutyramide, and the —NH(methyl) and —N(methyl)₂ derivatives of these amides.

The reactions described herein are photocatalytic reactions that take place in the presence of light, oxygen, and one or more photocatalysts (e.g., a tungstic acid or a salt thereof). Generally speaking, and without limitation, photocatalytic reactions involve the absorption of light by one or more reacting species in the presence of one or more photocatalysts that participate in the chemical reaction without being consumed.

The photocatalytic reactions described herein can be homogeneous, where the reactant(s) and the photocatalyst(s) exist in the same phase, or heterogeneous, where the photocatalyst(s) exist in a different phase from the reactant(s). The reactions may be conducted in a solvent, or without (i.e., in a neat condition). Preferably, long ultraviolet light and/or short visible light is used, for example, light in the wavelength range of 200-500 nm, preferably 250-450 nm, more preferably 300-425 nm, including 320, 340, 350, 360, 365, 370, 380, 390, 400, 410, and 420 nm. Any source of such wavelength(s) of light can be used, as can any suitable photoreaction vessel (batch reactor, flow reactor, etc.) that allows these wavelengths of light to irradiate the reactant(s), such as those constructed of borosilicate glass. A flow reactor and a bubble column reactor (batch or semi-batch) are preferable, for example, for scaling up the isobutyric acid (IBA) to methacrylic acid (MAA) conversion, especially when only low oxygen or air pressure is required. The irradiance can be varied within wide limits, for example, from 0.1 mW/cm² to 10,000 mW/cm², such as 1 mW/cm² to 1200 mW/cm² and including 10, 20, 40, 60, 80, 100, 200, 300, 400, 500, 600, 700, 800, 900, and 1000 mW/cm². A preferable range of irradiance is about 10 mW/cm² to 400 mW/cm².

The set up and initiation of the reactions described herein is well within the ability of one of ordinary skill in this art in view of the present description, as is the monitoring of reaction progress, including the identity and quantity of the products produced, by chromatography, NMR, etc., for example, using an internal standard. Reaction times can be varied within wide limits (e.g., from 15 min to 72 hours or more) depending on various reaction variables, such as the intensity of light, wavelength of light, concentration of reactant(s), reaction temperature (e.g., from −30 to 200 °C, including-20 °C, −10 °C, 0 °C, 10 °C, room temperature (20 °C), as well as 30, 40, 50, 60, 70, 80, and 90 °C, etc.), etc., all of which are within the ability of one of ordinary skill in this art in view of the present description.

When a solvent is used, its choice is not limited. Preferably, the solvent does not appreciably absorb the irradiated light, and it dissolves the reactant(s). Mixtures may be used. Examples include, e.g., acetonitrile, acetone, water, dichloromethane, dimethylformamide, dimethylsulfoxide, ethyl acetate, pyridine, tetrahydrofuran, etc. Preferred solvent includes acetonitrile and acetone.

When a solvent is used, the amount thereof is not particularly limited and can vary within wide limits. In view of the present disclosure, one of ordinary skill in the art is capable of determining the amount to be used based on reaction conditions, etc. For example, the amount of solvent used can be quantified on a volume/volume (v/v) basis where the reactant, e.g., isobutyric acid, and the solvent are both in liquid form at room temperature; and can also be used for reactants that are solid at room temperature by performing the measurement at a temperature above its and the solvent's melting points. Example v/v ratios of reactant/solvent include 1/99, 5/95, 10/90, 15/85, 20/80, 30/70, 40/60, 50/50, 60/40, 30/70, 80/20, 85/15, 90/10, 95/5, and 99/1. The amount of solvent used can also be quantified on a concentration (molar) basis. For example, concentrations of 0.01-30 M reactant based on reaction volume may be used, including, for example, 0.05, 0.1, 0.2, 0.3, 0.4, 0.5, 0.6, 0.7, 0.8, 0.9, 1, 2, 3, 4, 5, 6, 7, etc., M.

Oxygen is present in the reaction mixtures used herein. It can be provided in any form, and in multiple forms, such as in air present in the solvent (if any), in air present in the reaction chamber, in air added to the reaction chamber in intermittent or continuous fashion, in air used to pressurize the reaction vessel if conducting the reaction above normal pressure, etc. Of course, pure oxygen, or a mixture of oxygen with air and/or any other inert gas(ses) may be used in the same fashion as, or in addition to, the ways in which air may be used. If desired, the reaction vessel may be purged of gasses other than oxygen and maintained in a fashion such that only oxygen or a mixture of oxygen with one or more other selected gasses such as, e.g., nitrogen, helium, neon, argon, krypton, xenon, etc., in a selected ratio is present during reaction. Such methods are well within the skill of one of ordinary skill in this art in view of the present description. When pressure is used in the reactions herein, oxygen pressure can be in the range of about 1 atmosphere (0 psig) to 5000 psig, including, for example, 1, 5, 10, 15, 20, 25, 30, 35, 40, 45, 50, 75, 100, 250, 500, and 1000 psig; and preferably, 0-75 psig; and more preferably, 10-30 psig.

The present reactions are photocatalyzed in the presence of oxygen, meaning that the reactant(s) are irradiated in the presence of one or more photocatalysts. The photocatalyst, when a solvent is used in the reaction, is not necessarily soluble in the solvent.

The photocatalyst can be a tungstic acid or a salt thereof. Exemplary tungstic acids include H₂WO₄ and H₃PW₁₂O₄₀. Exemplary tungstic acid salts include tetra-n-butylammonium decatungstate (TBADT), sodium decatungstate (NaDT), calcium tungstate (CaWO₄), potassium tungstate (K₂WO₄), lithium tungstate (Li₂WO₄), sodium tungstate (Na₂WO₄), ammonium tungstate ((NH₄)₁₀H₂(W₂O₇)₆), cadmium tungstate (Cd₂WO₄), ammonium paratungstate ((NH₄)₁₀H₂(W₂O₇)₆·xH₂O), sodium polytungstate (3Na₂WO₄·9WO₃·H₂O), (Bu₄N)₃PW₁₂O₄₀, (NH₄)₆P₂W₁₈O₆₂, and (Bu₄N)₂W₆O₁₉. See, e.g., Angew. Chem. Int. Ed. Engl. 27, 1988, 1526-1527, incorporated herein by reference in entirety.

A preferred photocatalyst for use in the reactions described herein is a salt of the decatungstate anion (W₁₀O₃₂⁻⁴; “decatungstate”). Counterions therefor are not limited and include, e.g., sodium, lithium, potassium, iron, magnesium, calcium, strontium, barium, aluminum, tin, manganese, cobalt, lead, nickel, copper, silver, gold, zinc, mercury, phosphonium, imidazolium, pyrrolidinium, piperidinium, pyridinium, ammonium, and (C₁-C₈ alkyl) 1-4 substituted phosphonium, imidazolium, pyrrolidinium, piperidinium, pyridinium, and ammonium, where the substituents can be the same or different. A preferred decatungstate acid salt compound herein is tetrabutylammonium decatungstate (TBADT). See, e.g., Tzirakis et. al, Chem. Soc. Rev., 2009, 38, 2609-262 and Laudadio et al., Science, 2020 Jul. 3, 369 (6499): 92-96, both incorporated herein by reference in entirety. More than one decatungstate compound can be used to provide the decatungstate anion in the reactions described herein.

Other inorganic photocatalysts (e.g., metal chlorides) and organic photocatalysts (e.g., organic benzophenone derivatives), can also be used. Examples include benzophenone, bis(4-(trifluoromethyl)phenyl)methanone, bis(4-chlorophenyl)methanone, (4-methoxyphenyl)(4-(trifluoromethyl)phenyl)methanone, acetophenone, 9H-fluoren-9-one, phenanthrene-9,10-dione, pentacene-5,7,12,14-tetraone, 2-oxo-2-phenylacetic acid, 3a,5a-dihydropyrene-1,6-dione, anthracene-9,10-dione, 2-chloroanthraquinone, xanthone, thioxanthone, FeCl₃·6H₂O, FeCl₂·4H₂O, CuCl₂, and (TEA)₂CeCl₆.

The concentration or amount of photocatalyst used in the reactions described herein is not particularly limited and can vary within wide limits. In view of the present disclosure, one of ordinary skill in the art is capable of determining the amount to be used based on reaction conditions, etc. For example, concentrations of 0.01-30 mM decatungstate may be used, including, for example, 0.01, 0.05, 0.1, 0.2, 0.3, 0.4, 0.5, 0.6, 0.7, 0.8, 0.9, 1, 2, 3, 4, 5, 6, 7, 15, 20, 25, etc., mM. In cases where the photocatalyst does not dissolve completely or at all in the solvent, if any, and/or in the reactant(s), the amount of photocatalyst used is not limited. For example, it can be that amount that would produce a concentration noted above if it were dissolved. However, it can be more or less than this amount and can depend on the physical form of the catalyst, the amount of surface area exposed to the reaction medium, etc.

In a method of producing a compound of formula (3), a compound of formula (3′), or both, described herein, a compound of formula (1) is oxidized via a photocatalytic reaction, that is, is irradiated in the presence of oxygen, a photocatalyst (such as a tungstic acid and/or a salt thereof), and optionally a solvent, to produce at least one of compounds (3) and (3′):

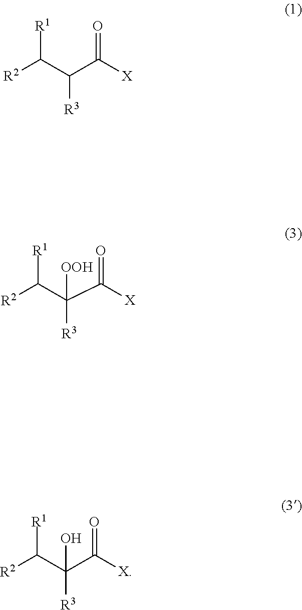

Compounds (3) and (3′) are useful intermediates for the production of compounds of formula (2), for example, through dehydration/dehydroperoxidation:

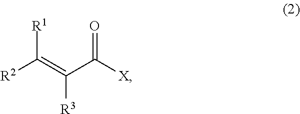

where compounds of formula (2) are α,β unsaturated carbonyl compounds.

For example, compounds of formula (3) can be converted to compounds of formula (3′) through reaction with trimethyl phosphite (a reductant), by heating, etc. Compounds of formula (3′) can be converted into compounds of formula (2) for example through dehydration, etc., using well known reactions, methods, etc., such as those described in EP 0 941 984 A2, U.S. Pat. No. 10,138,306 B1, and Pirmorade et al., ACS Sustainable Chem. Eng. 2017, 5, 1517-1527, all of which are incorporated herein by reference. Other catalytic, chemical, etc., methods of converting compounds of formula (3) and formula (3′) to compounds of formula (2) can be used.

In a preferred embodiment herein, and while not bound by theory, the inventive process of reacting compounds of formula (1) to prepare compounds of formula (3), (3′), and (2) is shown below using as an example the preparation of methacrylic acid (MAA) from isobutyric acid (IBA) in the presence of oxygen and the tetrabutylammonium decatungstate (TBADT) photocatalyst (a similar process could be shown for the preparation of methyl methacrylate (MMA), etc. from methyl isobutyrate (MIB), etc.):

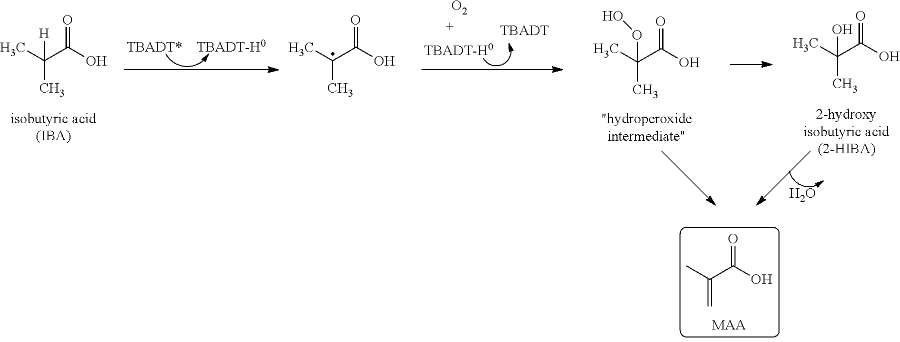

While not bound by theory, it is believed that, in the process, the C(sp³)-H bond of IBA (alpha to the carboxylic acid) is activated by using TBADT under irradiation to generate a carbon-centered radical; the carbon radical is then quenched by oxygen to form intermediates, i.e., 2-hydroperoxide isobutyric acid (2-IBA-OOH) and/or 2-hydroxy-isobutyric acid (2-HIBA); the intermediates can then undergo a dehydroperoxidation process and/or a dehydration process to form MAA. Side products can include acetone, acetic acid, and formic acid.

Products of the reactions herein can be isolated by conventional procedures well known to those of ordinary skill, including distillation, chromatography, filtration, extraction, etc.

As is understood by one of ordinary skill in the art, reaction variables, such as the type of photocatalyst, the amount of photocatalyst added to the reaction mixture, oxygen pressure, irradiance, temperature, solvent, and reaction time, all can impact the photocatalytic processes described herein.

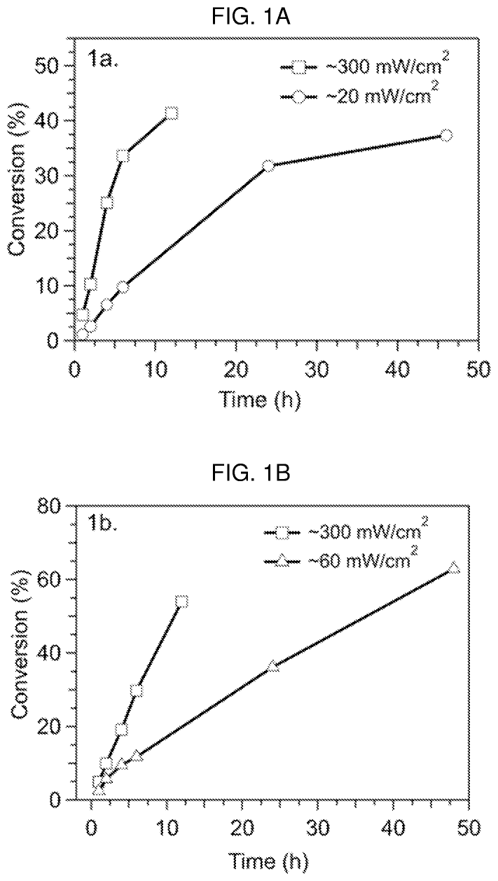

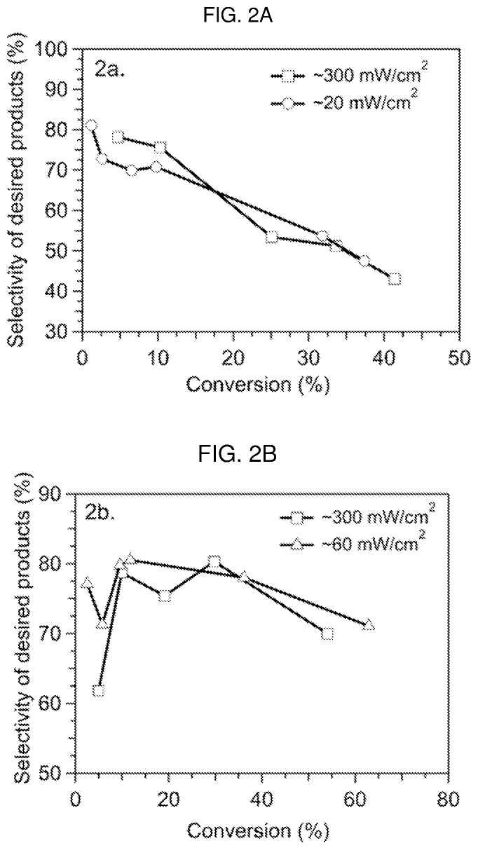

The reaction rate is highly dependent on irradiance. Increasing irradiance generally increases the reaction rate. For example, as shown in FIGS. 1A-1B, an irradiance of about 300 mW/cm² drastically increased the conversion rates for both IBA and MIB as compared with a low irradiance of about 20 or 60 mW/cm². The higher reaction rates due to a higher irradiance did not result in lower selectivity of the desired products for both IBA and MIB; instead, as shown in FIGS. 2A-2B, the selectivity was comparable at the same conversion while different irradiances.

Reasonable selectivity of the desired products is generally maintained at high conversion for MIB, as shown in FIG. 2B. However, as shown in FIG. 2A, selectivity of the desired products tends to become lower as conversion increases for IBA.

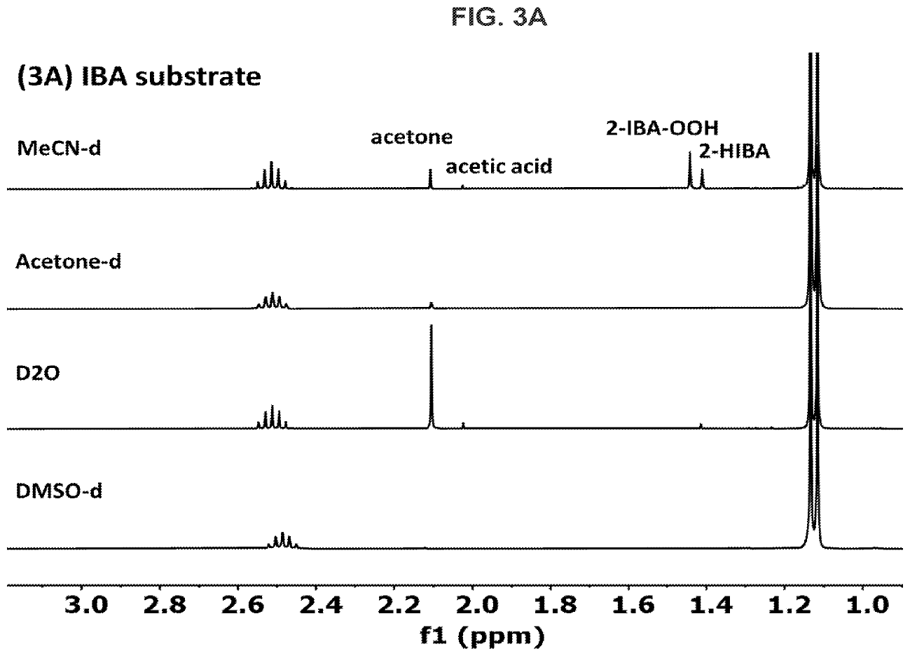

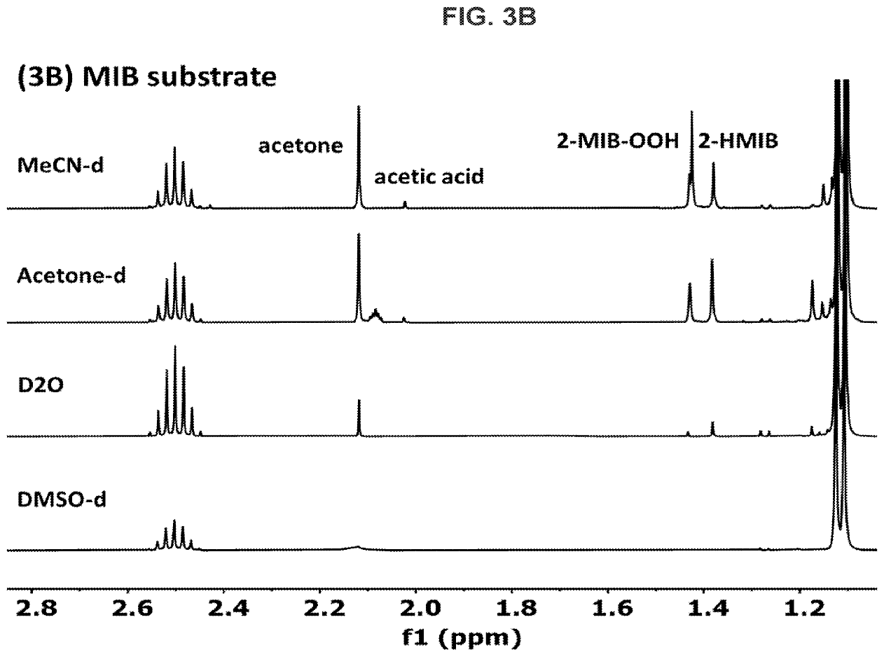

The choice of a solvent also affects the conversions of reactants and the selectivity of desired products. In one embodiment, four solvents were evaluated—acetonitrile (MeCN), acetone, water, and dimethylsulfoxide (DMSO)—in preparing MAA from IBA and in preparing MMA from MIB in the presence of oxygen and TBADT. For ease of product quantification and identification, deuterated solvents, i.e., MeCN-d₃, acetone-d₆, D₂O, and DMSO-d₆, were used. TBADT is easily soluble in MeCN-d₃ and DMSO-d₆, but not quite soluble in acetone-d₆ and D₂O. As shown in FIGS. 3A and 3B, it was found that MeCN-d₃ and acetone-do gave good IBA or MIB conversions as compared to D₂O and DMSO-d₆. Compared to MeCN-d₃, acetone-d₆ showed higher conversion of the reactants; however, the selectivity of desired products of hydroperoxide and hydroxy compounds using acetone-de was lower. In addition, in this study, high solvent ratio (e.g., 90%) generally gave higher conversion.

Among the photocatalysts evaluated for the processes described herein, a decatungstate, such as TBADT, exhibits superior performance in terms of conversion and total selectivity of desired products. High amounts of photocatalysts may have solubility issues when a solvent is used in a reaction. Thus, excess photocatalyst is not desirable. In converting IBA or MIB, for example, a TBADT loading of 0.5 mM-2 mM can be used in certain embodiments.

An excessively low oxygen environment gives low conversion and selectivity, while high oxygen pressure does not necessarily result in high conversion. In certain embodiments, air (containing about 80% of N₂ and about 20% of O₂) gives better performance than pure oxygen. In certain embodiments, higher oxygen pressure leads to decreased conversion. In converting IBA or MIB, the oxygen pressure is preferably from one atmosphere (0 psig) to 75 psig; more preferably, 0-40 psig; and even more preferably, 0-20 psig.

In certain embodiments, the reactions described herein are less sensitive to temperature when v/v ratios of reactants (such as IBA, MIB) to solvent is less than 50/50. In certain embodiments, the conversion of reactants may be increased when temperature is increased. In certain embodiments, high temperatures may tend to increase the selectivity of side products; while in certain embodiments, lower temperatures tend to improve selectivity toward desired products.

In certain embodiments, with the same irradiance, higher conversions of the reactants (95% or more) can be achieved with longer reaction time (e.g., 65, 70, 80, 90, 100, 120, 140, etc. hours or more). As described above, higher irradiance significantly shortens the reaction time to achieve a high conversion of the reactants.

In addition, in certain embodiments, photoreactions using flow reactors achieve better performance (higher product selectivity, higher selectivity toward desired products, higher conversion, etc. at lower irradiance with a shorter reaction time) than those using batch reactors under similar reaction conditions.

### EXAMPLES

### Typical Reaction Procedure, Reaction Setup, and Result Evaluation

When preparing the examples using batch reactors, typically, a glass tube (pressure rating=150 psi, Ace glass part #8648-62, 9 ml capacity) was charged with a stir bar and a photocatalyst. Reactant (IBA or MIB) (containing 1,4-bis(trifluoromethyl)benzene as an internal standard to quantify the conversion of the reactant and the selectivity of desired and side products) and solvent were pipetted into the glass tube before the reaction. These reagents were introduced under ambient atmosphere without degassing so that the glass tube initially contained 1 atm of air (about 20% O₂ and about 80% N₂). The glass tube was then sealed; and pure oxygen or additional air was introduced. The glass tube was irradiated with Kessil PR160L-370 LED light (370 nm) or Lumidox® Gen II 96-Well LED Arrays by Analytical Sales and Services (365 nm). The reaction temperature was maintained by circulating chiller solution around the glass tube. The reaction mixtures were agitated and irradiated until the reaction time was up.

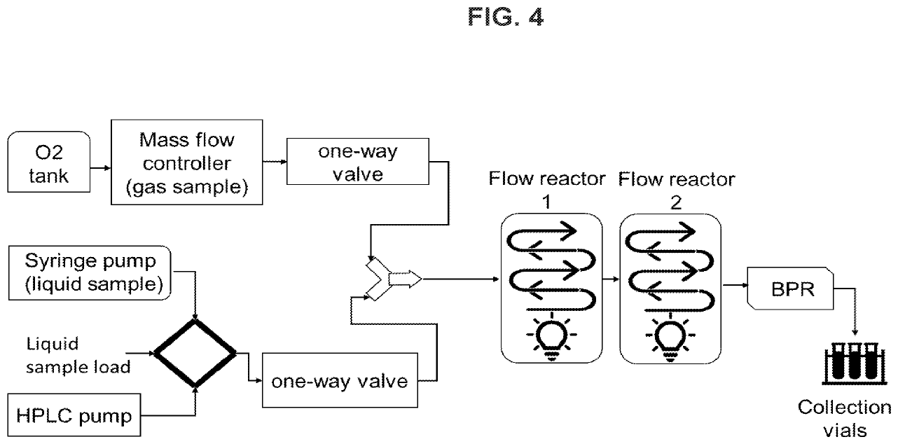

An exemplified setup when a flow photoreactor was used for the photoreaction is shown in FIG. 4, where two flow photoreactors irradiated with LED lights were connected in series and the liquid sample (solvent and reactant (IBA or MIB) containing 1,4-bis(trifluoromethyl)benzene as an internal standard) and the gas sample (oxygen and/or air) were dosed continuously with a syringe pump and mass flow controller, respectively. The liquid and gas samples were mixed in a Y-type mixer to create well-defined gas-liquid segments before going through the photoreactors. The gas/liquid ratio (G/L ratio) was controlled by gas and liquid flow rates. The system pressure was regulated by a back pressure regulator (BPR) positioned after the photoreactors. At the conclusion of the reaction, samples were collected into an ice-bath cooled vial containing d-acetonitrile.

Different setups by adjusting, for example, the LED output and the distance between LED lights and the glass tube, were made to achieve various irradiances. For instance, Lumidox® Gen II 96-Well LED Arrays at a close distance of about 0.5 cm away from the glass tube achieved an irradiance of about 300 mW/cm²; Kessil PR160L-370 LED light positioned about 10 cm away from the glass tube achieved an irradiance of about 60 mW/cm²; and Kessil PR160L-370 LED light positioned about 12 cm away from the glass tube achieved an irradiance of about 20 mW/cm². Higher irradiance (e.g., 400 and 900 mW/cm²) could be achieved by using higher LED output and/or arranging the LED array in closer proximity to the glass tube.

The photocatalytic process was evaluated by measuring the conversion of the reactant, total selectivity, selectivity toward desired products of hydroperoxide and hydroxy compounds, selectivity of side products of acetic acid and acetone, and total turnover number (TON) of the photocatalyst. A high TON represents good stability of a photocatalyst.

Reactions with Different Conditions

In one embodiment using TBADT as the photocatalyst, results of IBA with different reaction conditions (Examples 1-17) and results of MIB with different reaction conditions (Examples 18-35) using batch reactors are shown in Table 1 and Table 2, respectively. In these examples, deuterated acetonitrile (d-MeCN) or deuterated acetone (d-acetone) was used as the solvent; and the irradiance (measured by Ophir Photonics (Parts number: 7Z02480 PD300RM-8W and 7Z01565 StarLite) unless otherwise indicated) ranged from 20 to 60 mW/cm² except for Examples 1 and 17 in Table 1 and Example 18 in Table 2. Example 1 in Table 1 and Example 18 in Table 2 were prepared at an irradiance of 300 mW/cm²; and Example 17 in Table 1 was prepared at an irradiance of about 100 mW/cm² (estimated from actinometry). In addition, air instead of pure oxygen, as indicated in Table 1, was used as the oxidant in preparing Example 17.

From the examples, certain observations can be made regarding this embodiment concerning the effects on the photocatalytic reaction run of the loading of TBADT, solvent, the amount of solvent used in a reaction mixture, oxygen pressure, temperature, and reaction time.

In particular, the conversion of IBA or MIB did not depend heavily on the loading of TBADT, which also indicated the stability of TBADT. Generally, 0.5 mM or 2 mM of TBADT gave good and comparable performance. However, lesser performance (as indicated by, for example, a low TON) was observed at a high loading of 4 mM (see, e.g., Ex. 14 in Table 1 and Ex. 34 in Table 2) due possibly to poor solubility of the photocatalyst in the solvent. Generally, the stability of TBADT was good, as evidenced by an increased conversion of the reactant and an increased TON at an increasing reaction time (see, e.g., Ex. 27 vs. Ex. 29 in Table 2).

When a solvent was used, the conversion of the reactant was related to the reactant concentration in the solution. From the comparison of, for example, Ex. 4 vs. Ex. 5 in Table 1 and Ex. 22 vs. Ex. 23, Ex. 25 vs. Ex. 26, and Ex. 29 vs. Ex. 30 in Table 2, it can be seen that a higher solvent ratio generally gave a higher conversion of the reactant.

The conversion and selectivity also depended on the solvent. Both acetonitrile and acetone could promote higher conversion of IBA and a higher conversion of MIB as compared with water and dimethylsulfoxide. Between acetonitrile and acetone, d-MeCN showed higher selectivity of desired products (i.e., 2-HIBA and 2-IBA-OOH) for IBA than d-acetone, as shown by the comparison between Exs. 10 and 15 and between Exs. 11 and 16 in Table 1. The same observation can be made for MIB (see, e.g., Ex. 27 vs. Ex. 35 in Table 2).

The effect of temperature on MIB conversion in this embodiment appeared related to the loading of the reactant. For 50% MIB/50% d-MeCN solution, the conversion significantly increased from 10.7% (Ex. 22) to 41.4% (Ex. 28) when temperature was increased from 10 °C to 50 °C. However, when more solvent was present relative to the reactant (e.g., 10% MIB/90% d-MeCN solution), the reaction appeared less sensitive to temperature change. In addition, a low temperature, such as 0 °C and 10 °C, appeared to improve selectivity toward the desired products of 2-HMIB and 2-MIB-OOH (e.g., Exs. 18-23 in Table 2).

In the case of IBA, temperature did not appear to have much effect on the conversion in this embodiment, regardless of the loading of the reactant relative to the solvent. However, similar to the case of MIB, a low temperature (e.g., 0 °C) generally tended to have a higher selectivity toward desired products of 2-HIBA and 2-IBA-OOH than a high temperature (e.g., Exs. 1 and 2 in Table 1).

An excessively low oxygen environment tended to give low conversion and low selectivity in this embodiment. However, when sufficient oxygen was supplied initially or when oxygen was supplied continuously, oxygen pressure did not seem to appreciably affect the results for both IBA and MIB. For example, the conversion and total selectivity were almost the same with oxygen pressures of 10 psig and 20 psig (see, e.g., Ex. 6 vs. Ex. 10 and Ex. 7 vs. Ex. 11 in Table 1; Ex. 24 vs. Ex. 31, Ex. 25 vs. Ex. 32, and Ex. 27 vs. Ex. 33 in Table 2). It was also found that air generally performed better than pure oxygen, especially in terms of the selectivity of desired products, as evidenced by Ex. 17 in Table 1.

The examples also show that higher conversions could be achieved with longer reaction time (see, Ex. 27 vs. Ex. 29 in Table 2), which indicates a robustness of the TBADT photocatalyst.

TABLE 1
Summary of results of IBA with different reaction conditions

| | | | | | | | Results | | | | | | |
|---|---|---|---|---|---|---|---|---|---|---|---|---|---|
| | | Reaction Conditions | | | | | | Total | Selec. | Selec. | Selec. | Selec. | |
| Ex. | Run | TBADT | Solvent | P_{oxygen} | T | Time | Conv. | Selec. | of 2-HIBA | of 2-IBA- | of acetic | of acetone | Total |
| # | # | (mM) | (% v/v) | (psig) | (°C) | (h) | (%) | (%) | (%) | OOH (%) | acid (%) | (%) | TON |
| 1 | jsr-yl- | 2 | d-MeCN | 20 | 0 | 6 | 33.6 | 88.9 | 5.3 | 45.9 | 6.7 | 26.9 | 155 |
| | 103-1 | | (90% v/v) | | | | | | | | | | |
| 2 | jsr-2ec- | 2 | d-MeCN | 20 | 0 | 16 | 34.2 | 91.1 | 10.5 | 61.0 | 4.6 | 15.0 | 165 |
| | 67-a1 | | (90% v/v) | | | | | | | | | | |
| 3 | jsr-2ec- | 0.5 | d-MeCN | 20 | 10 | 16 | 15.6 | 69.3 | 7.4 | 35.6 | 3.5 | 22.8 | 1139 |
| | 57-a1 | | (50% v/v) | | | | | | | | | | |
| 4 | jsr-2ec- | 2 | d-MeCN | 20 | 10 | 16 | 24.5 | 79.4 | 9.1 | 22.8 | 5.6 | 42.0 | 508 |
| | 57-a3 | | (50% v/v) | | | | | | | | | | |
| 5 | jsr-2ec- | 2 | d-MeCN | 20 | 10 | 16 | 49.2 | 74.8 | 7.5 | 17.5 | 8.4 | 41.4 | 195 |
| | 57-a5 | | (90% v/v) | | | | | | | | | | |
| 6 | jsr-2ec | 0.5 | d-MeCN | 20 | 30 | 16 | 11.8 | 103.3 | 19.3 | 44.0 | 6.5 | 33.5 | 1259 |
| | 51-a1 | | (50% v/v) | | | | | | | | | | |
| 7 | jsr-2ec- | 2 | d-MeCN | 20 | 30 | 16 | 19.9 | 94.3 | 13.2 | 10.4 | 9.5 | 61.1 | 483 |
| | 51-a3 | | (50% v/v) | | | | | | | | | | |
| 8 | jsr-2ec- | 2 | d-MeCN | 20 | 50 | 16 | 43.4 | 87.7 | 23.4 | 19.3 | 13.8 | 31.2 | 193 |
| | 55-a5 | | (90% v/v) | | | | | | | | | | |
| 9 | jsr-yl- | 0.5 | d-MeCN | 20 | 50 | 65 | 78.1 | 82.2 | 0.2 | 0.3 | 23.3 | 58.4 | 1306 |
| | 93-b4 | | (90% v/v) | | | | | | | | | | |
| 10 | jsr-yl- | 0.5 | d-MeCN | 10 | 30 | 16 | 13.2 | 77.0 | 14.7 | 45.2 | 2.7 | 14.4 | 1020 |
| | 89-a1 | | (50% v/v) | | | | | | | | | | |
| 11 | jsr-yl- | 2 | d-MeCN | 10 | 30 | 16 | 20.2 | 68.9 | 12.7 | 11.7 | 4.3 | 40.3 | 348 |
| | 89-a3 | | (50% v/v) | | | | | | | | | | |
| 12 | jsr-yl- | 0.5 | d-MeCN | 10 | 50 | 16 | 18.4 | 102.6 | 10.7 | 5.7 | 13.5 | 72.8 | 1894 |
| | 91-5a | | (50% v/v) | | | | | | | | | | |
| 13 | jsr-yl- | 2 | d-MeCN | 10 | 50 | 16 | 25.8 | 89.3 | 10.7 | 11.9 | 7.4 | 59.3 | 577 |
| | 91-6a | | (50% v/v) | | | | | | | | | | |
| 14 | jsr-yl- | 4 | d-MeCN | 10 | 30 | 16 | 5.8 | 208.3 | 48.0 | 94.2 | 15.5 | 50.5 | 151 |
| | 89-a5 | | (50% v/v) | | | | | | | | | | |
| 15 | jsr-yl- | 0.5 | d-acetone | 10 | 30 | 16 | 14.5 | 62.8 | 4.1 | 1.5 | 5.4 | 51.9 | 915 |
| | 89-a2 | | (50% v/v) | | | | | | | | | | |
| 16 | jsr-yl- | 2 | d-acetone | 10 | 30 | 16 | 8.6 | 98.5 | 7.1 | 6.0 | 9.5 | 75.8 | 212 |
| | 89-a4 | | (50% v/v) | | | | | | | | | | |
| 17 | jsrb-yl- | 2 | d-MeCN | 5 (air) | 0 | 4 | 17.1 | 104.4 | 4.8 | 66.1 | 6.5 | 26.9 | 94 |
| | 150 | | (90% v/v) | | | | | | | | | | |

TABLE 2
Summary of results of MIB with different reaction conditions

| | | | | | | | Results | | | | | | |
|---|---|---|---|---|---|---|---|---|---|---|---|---|---|
| | | Reaction Conditions | | | | | | Total | Selec. | Selec. | Selec. | Selec. | |
| Ex. | Run | TBADT | Solvent | P_{oxygen} | T | Time | Conv. | Selec. | of 2-HMIB | of 2-MIB- | of acetic | of acetone | Total |
| # | # | (mM) | (% v/v) | (psig) | (°C) | (h) | (%) | (%) | (%) | OOH (%) | acid (%) | (%) | TON |
| 18 | jsr-yl- | 2 | d-MeCN | 20 | 0 | 12 | 54.0 | 82.8 | 9.5 | 60.5 | 4.3 | 8.4 | 177 |
| | 103-2 | | (90% v/v) | | | | | | | | | | |
| 19 | jsr-2ec- | 2 | d-MeCN | 20 | 0 | 16 | 54.5 | 85.0 | 9.1 | 61.4 | 4.8 | 9.7 | 174 |
| | 63-b4 | | (90% v/v) | | | | | | | | | | |
| 20 | jsr-yl- | 2 | d-MeCN | 20 | 0 | 22 | 53.6 | 84.1 | 7.2 | 66.1 | 3.9 | 6.9 | 189 |
| | 101-4 | | (90% v/v) | | | | | | | | | | |
| 21 | jsr-2ec- | 0.5 | d-MeCN | 20 | 10 | 16 | 9.8 | 83.1 | 5.7 | 73.4 | 0.7 | 3.3 | 649 |
| | 57-b1 | | (50% v/v) | | | | | | | | | | |
| 22 | jsr-2ec- | 2 | d-MeCN | 20 | 10 | 16 | 10.7 | 83.0 | 6.5 | 71.1 | 1.5 | 3.9 | 178 |
| | 57-b3 | | (50% v/v) | | | | | | | | | | |
| 23 | jsr-2ec- | 2 | d-MeCN | 20 | 10 | 16 | 58.8 | 79.9 | 7.1 | 61.2 | 4.0 | 7.6 | 188 |
| | 57-b5 | | (90% v/v) | | | | | | | | | | |
| 24 | jsr-2ec- | 0.5 | d-MeCN | 20 | 30 | 16 | 12.8 | 79.4 | 6.7 | 64.3 | 1.2 | 7.3 | 811 |
| | 51-b1 | | (50% v/v) | | | | | | | | | | |
| 25 | jsr-2ec- | 2 | d-MeCN | 20 | 30 | 16 | 17.3 | 82.3 | 7.1 | 67.9 | 1.7 | 5.6 | 284 |
| | 51-b3 | | (50% v/v) | | | | | | | | | | |
| 26 | jsr-2ec- | 2 | d-MeCN | 20 | 30 | 16 | 74.5 | 73.1 | 4.7 | 49.5 | 6.7 | 12.1 | 217 |
| | 51-b5 | | (90% v/v) | | | | | | | | | | |
| 27 | jsr-2ec- | 0.5 | d-MeCN | 20 | 50 | 16 | 21.1 | 68.9 | 9.7 | 36.5 | 3.8 | 18.9 | 1201 |
| | 47-b1 | | (50% v/v) | | | | | | | | | | |
| 28 | jsr-2ec- | 2 | d-MeCN | 20 | 50 | 16 | 41.4 | 72.5 | 19.8 | 2.8 | 8.1 | 41.8 | 604 |
| | 55-b3 | | (50% v/v) | | | | | | | | | | |
| 29 | jsr-yl- | 0.5 | d-MeCN | 20 | 50 | 65 | 40.1 | 65.5 | 15.3 | 5.4 | 7.8 | 37.0 | 2110 |
| | 93-a2 | | (50% v/v) | | | | | | | | | | |
| 30 | jsr-yl- | 0.5 | d-MeCN | 20 | 50 | 65 | 92.5 | 69.6 | 7.7 | 1.7 | 23.6 | 36.7 | 1035 |
| | 93-a4 | | (90% v/v) | | | | | | | | | | |
| 31 | jsr-yl- | 0.5 | d-MeCN | 10 | 30 | 16 | 18.7 | 75.3 | 6.7 | 61.1 | 1.3 | 6.3 | 1120 |
| | 89-b1 | | (50% v/v) | | | | | | | | | | |
| 32 | jsr-yl- | 2 | d-MeCN | 10 | 30 | 16 | 16.2 | 79.7 | 8.9 | 60.8 | 1.9 | 8.2 | 258 |
| | 89-b3 | | (50% v/v) | | | | | | | | | | |
| 33 | jsr-2ec- | 0.5 | d-MeCN | 10 | 50 | 16 | 29.8 | 66.0 | 13.8 | 16.8 | 6.2 | 29.2 | 1631 |
| | 49-b3 | | (50% v/v) | | | | | | | | | | |
| 34 | jsr-yl- | 4 | d-MeCN | 10 | 30 | 16 | 17.7 | 71.9 | 13.3 | 37.9 | 3.7 | 17.1 | 126 |
| | 89-b5 | | (50% v/v) | | | | | | | | | | |
| 35 | jsr-2ec- | 0.5 | d-acetone | 20 | 50 | 16 | 34.5 | 62.1 | 20.1 | 6.4 | 5.2 | 30.4 | 1771 |
| | 47-b2 | | (50% v/v) | | | | | | | | | | |

### Reactions Using Different Photocatalysts

In other embodiments, in addition to TBADT, four organic photocatalysts, i.e., benzophenone, xanthone, thioxanthone, and 2-chloroanthraquinone, and an inorganic photocatalyst, FeCl₃·6H₂O, were evaluated for activation of the C(sp³)-H bond of IBA or MIB.

In these examples, each photocatalyst in an amount of 2 mM was added to the reactant solution in deuterated acetonitrile (d-MeCN, 90% v/v). Reactions were conducted in a Hepatochem batch reactor with 370 nm LED light irradiation (irradiance of 20-60 mW/cm²) and 20 psig of O₂ at 0 °C for 16 hours. The results were analyzed by 1H-NMR and shown in Table 3 (Examples 2 and 36-40) and Table 4 (Examples 41-46) for IBA and MIB, respectively.

TABLE 3
Results of IBA with different photocatalysts

| | | | | Selec. of | Selec. of | Selec. of | Selec. of | |
|---|---|---|---|---|---|---|---|---|
| | | | Conv. | 2-HIBA | 2-IBA- | acetic | acetone | Total |
| Ex. # | Run # | Photocatalyst | (%) | (%) | OOH (%) | acid (%) | (%) | TON |
| 2 | jsr-2ec- | TBADT | 34.2 | 10.5 | 61.0 | 4.6 | 15.0 | 165 |
| | 67-a1 | | | | | | | |
| 36 | jsr-2ec- | Benzophenone | 0 | 0 | 0 | 0 | 0 | 0 |
| | 67-a2 | | | | | | | |
| 37 | jsr-2ec- | Xanthone | 0 | 0 | 0 | 0 | 0 | 0 |
| | 67-a3 | | | | | | | |
| 38 | jsr-2ec- | thioxanthone | 0 | 0 | 0 | 0 | 0 | 0 |
| | 67-a4 | | | | | | | |
| 39 | jsr-2ec- | 2- | 4.6 | 6.5 | 15.3 | 7.7 | 45.2 | 18 |
| | 67-a5 | chloroanthraquinone | | | | | | |
| 40 | jsr-2ec- | FeCl₃•6H₂O | 40.0 | 14.1 | 2.5 | 12.3 | 16.4 | 96 |
| | 67-a6 | | | | | | | |

TABLE 4
Results of MIB with different photocatalysts

| | | | | Selec. of | Selec. of | Selec. of | Selec. of | |
|---|---|---|---|---|---|---|---|---|
| | | | Conv. | 2-HMIB | 2-MIB- | acetic | acetone | Total |
| Ex. # | Run # | Photocatalyst | (%) | (%) | OOH (%) | acid (%) | (%) | TON |
| 41 | jsr-2ec- | TBADT | 23.9 | 6.2 | 72.3 | 4.7 | 5.9 | 84 |
| | 67-b1 | | | | | | | |
| 42 | jsr-2ec- | Benzophenone | 0.2 | 28.6 | 25.0 | 14.3 | 35.7 | 1 |
| | 67-b2 | | | | | | | |
| 43 | jsr-2ec- | Xanthone | 0.7 | 7.6 | 7.1 | 3.5 | 9.4 | 1 |
| | 67-b3 | | | | | | | |
| 44 | jsr-2ec- | thioxanthone | 1.5 | 4.3 | 2.9 | 1.6 | 7.8 | 1 |
| | 67-b4 | | | | | | | |
| 45 | jsr-2ec- | 2- | 8.2 | 12.5 | 5.9 | 4.1 | 28.9 | 9 |
| | 67-b5 | chloroanthraquinone | | | | | | |
| 46 | jsr-2ec- | FeCl₃•6H₂O | 46.1 | 8.2 | 9.2 | 5.1 | 13.4 | 51 |
| | 67-b6 | | | | | | | |

As compared to TBADT, FeCl₃·6H₂O showed high conversion of both IBA and MIB; however, the selectivity of the desired versus side products was generally low.

### Comparison Between Reactions Using Batch Reactors and Flow Reactors

The performance using flow reactors versus batch reactors for both IBA and MIB with similar reaction conditions was evaluated by comparing Examples 47 and 48 for IBA and Examples 49 and 50 for MIB (Table 5).

The reaction solution of these examples contained 90% of d-acetonitrile and 10% of IBA or MIB. All the reactions occurred using a light of irradiation wavelength of 365 nm and TBADT (2 mM) as the photocatalyst at a temperature of 25 °C and an oxygen pressure of 20 psig. When flow reactors were used (i.e., Examples 47 and 49), a G/L ratio of 10 was applied to ensure that sufficient amount of O₂ was introduced; the gas flow rate was 0.1 mL/min; and the liquid flow rate was 0.01 mL/min. The conversion of the reactant (Conv. (%)) and selectivity toward desired products (Selectivity (%)) were quantified by 1H-NMR.

TABLE 5
Performance using flow reactors vs. batch reactors

| Ex. | Run | | | Time | Irradiance | Conv. | Selectivity |
|---|---|---|---|---|---|---|---|
| # | # | Reactant | Setup | (min) | (mW/cm²) | (%) | (%) |
| 47 | jsrf- | IBA | flow | 40 | 400 | 12.6 | 54.3 |
| | ec-51 | | | | | | |
| 48 | jsrb- | | batch | 60 | 900 | 15.3 | 39.0 |
| | yl-143 | | | | | | |
| 49 | jsrf- | MIB | flow | 40 | 400 | 30.2 | 42.5 |
| | ec-39 | | | | | | |
| 50 | jsrb- | | batch | 60 | 900 | 21.2 | 44.3 |
| | gq-15 | | | | | | |

Generally speaking, flow reactors outperform batch reactors where higher conversion of the reactant and/or higher selectivity toward desired products are obtained with lower irradiance in shorter reaction time.

As an alternative to a flow reactor examined here, a bubble column (batch or semi-batch) can be considered, for example, for scaling up IBA to MAA conversion, especially when only low O₂ or air pressure is required.

Considering the balance of factors, such as conversion of the reactant, selectivity of desired products, irradiance, and reaction time, Examples 1, 2, 17-20, and 49 as described above could be mentioned.

Preferred embodiments that are fully described and which are capable of being practiced by one of ordinary skill in the art based on the present description include:
1. A method, comprising irradiating a compound of formula (1) with 1 mW/cm² to 10,000 mW/cm² of irradiance having a wavelength of 200-500 nm in the presence of oxygen and a photocatalyst:

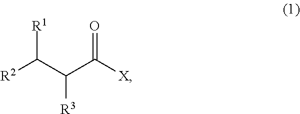

wherein:
R¹, R², and R³ each independently represent a hydrogen atom or an organic group having 1-20 carbon atoms; and
X represents a monovalent group represented by —OR^{a} or —NR^{b}R^{c}, where R^{a}, R^{b}, and R^{c} each independently represent a hydrogen atom or an organic group having 1-20 carbon atoms, and wherein optionally but preferably said irradiating causes the compound of formula (1) to change and produce at least one compound different from the compound of formula (1).
2. A method of preparing a compound of formula (3), a compound of formula (3′), or both from a compound of formula (1), the method comprising:
oxidizing the compound of formula (1) by a photocatalytic reaction in a presence of oxygen and a photocatalyst:

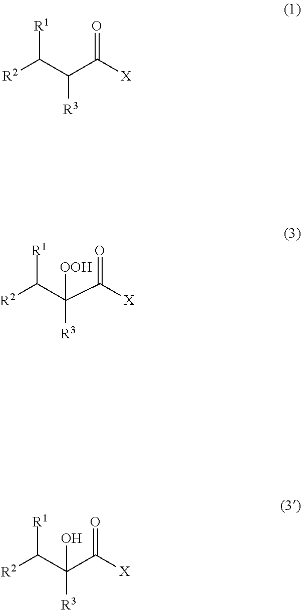

wherein:
R¹, R², and R³ each independently represent a hydrogen atom or an organic group having 1-20 carbon atoms; and
X represents a monovalent group represented by —OR^{a} or —NR^{b}R^{c}, where R^{a}, R^{b}, and R^{c} each independently represent a hydrogen atom or an organic group having 1-20 carbon atoms.
3. A method of preparing a compound of formula (2), the method comprising:
preparing a compound of formula (3) and/or a compound of formula (3′) by the method according to embodiment 2, and
converting the compound of formula (3) and/or the compound of formula (3′) to the compound of formula (2):

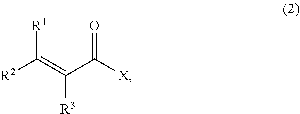

wherein R¹, R², R³, and X are the same as those contained in the compound of formula (1).
4. The method according to embodiments 1-3, wherein the compound of formula (1) is at least one selected from the group consisting of propionic acid, butyric acid, pentanoic acid, hexanoic acid, isobutyric acid, 2-methylbutyric acid, 2-methylpentanoic acid, 2-methylhexanoic acid, and a methyl ester thereof.
5. The method according to embodiment 3, wherein the compound of formula (1) is isobutyric acid and the compound of formula (2) is methacrylic acid.
6. The method according to embodiment 3, wherein the compound of formula (1) is methyl isobutyrate and the compound of formula (2) is methyl methacrylate.
7. The method according to embodiments 1-6, wherein the photocatalyst comprises at least one selected from the group consisting of an inorganic photocatalyst and an organic photocatalyst.
8. The method according to embodiments 1-7, wherein the photocatalyst comprises at least one inorganic photocatalyst selected from the group consisting of a tungstic acid and a salt of a tungstic acid.
9. The method according to embodiments 1-8, wherein the photocatalyst comprises at least one inorganic photocatalyst selected from the group consisting of tetrabutylammonium decatungstate, sodium decatungstate, calcium tungstate, potassium tungstate, lithium tungstate, sodium tungstate, ammonium tungstate, cadmium tungstate, ammonium paratungstate, sodium polytungstate, (Bu₄N)₃PW₁₂O₄₀, (NH₄)₆P₂W₁₈O₆₂, (Bu₄N)₂W₆O₁₉, FeCl₃·6H₂O, FeC₁₂·4H₂O, CuCl₂, and (TEA)₂CeCl₆.
10. The method according to embodiments 1-9, wherein the photocatalyst comprises a decatungstate.
11. The method according to embodiments 1-10, wherein the photocatalyst comprises tetrabutylammonium decatungstate.
12. The method according to embodiments 1-11, wherein the photocatalyst comprises at least one organic photocatalyst selected from the group consisting of benzophenone, bis(4-(trifluoromethyl)phenyl)methanone, bis(4-chlorophenyl)methanone, (4-methoxyphenyl) (4-(trifluoromethyl)phenyl)methanone, acetophenone, 9H-fluoren-9-one, phenanthrene-9,10-dione, pentacene-5,7,12,14-tetraone, 2-oxo-2-phenylacetic acid, 3a,5a-dihydropyrene-1,6-dione, anthracene-9,10-dione, 2-chloroanthraquinone, xanthone, and thioxanthone.
13. The method according to embodiments 1-12, wherein an amount of the photocatalyst is 0.5 mM or more and less than 4 mM.
14. The method according to embodiments 1-13, wherein the photocatalytic reaction occurs with irradiation from a light of a wavelength ranging from 300 nm to 425 nm and an irradiance ranging from 10 mW/cm² to 400 mW/cm².
15. The method according to embodiments 1-14, wherein the photocatalytic reaction occurs at an oxygen pressure ranging from 0 psig to 20 psig.
16. The method according to embodiments 1-15, wherein the photocatalytic reaction occurs in the presence of a solvent.
17. The method according to embodiments 1-16, wherein the solvent comprises at least one of acetonitrile and acetone.

The above written description of the invention provides a manner and process of making and using it such that any person of ordinary skill in the relevant art is enabled to make and use the same, this enablement being provided in particular for the subject matter of the appended claims, which make up a part of the original description. This description is provided in the context of a particular application and its requirements. Various modifications to the embodiments will be readily apparent to those of ordinary skill in the relevant art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A method, comprising irradiating a compound of formula (1) with irradiance of 1 mW/cm² to 10,000 mW/cm² having a wavelength of 200-500 nm in the presence of oxygen and a photocatalyst: 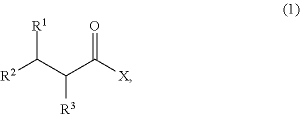
wherein:
R¹, R², and R³ each independently represent a hydrogen atom or an organic group having 1-20 carbon atoms; and
X represents a monovalent group represented by —OR^{a} or —NR^{b}R^{c}, where R^{a}, R^{b}, and R^{c} each independently represent a hydrogen atom or an organic group having 1-20 carbon atoms.

2. A method of preparing a compound of formula (3), a compound of formula (3′), or both from a compound of formula (1), the method comprising:
oxidizing the compound of formula (1) by a photocatalytic reaction in a presence of oxygen and a photocatalyst: 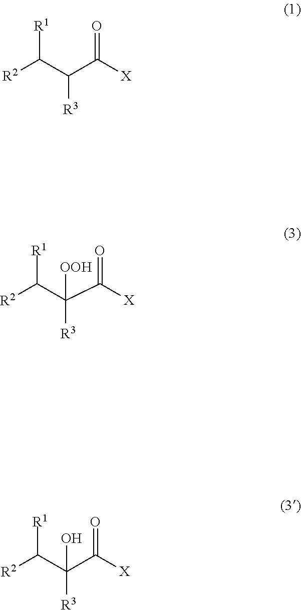
wherein:
R¹, R², and R³ each independently represent a hydrogen atom or an organic group having 1-20 carbon atoms; and
X represents a monovalent group represented by —OR^{a} or —NR^{b}R^{c}, where R^{a}, R^{b}, and R^{c} each independently represent a hydrogen atom or an organic group having 1-20 carbon atoms.

3. A method of preparing a compound of formula (2), the method comprising:
preparing a compound of formula (3) and/or a compound of formula (3′) by the method according to claim 2, and
converting the compound of formula (3) and/or the compound of formula (3′) to the compound of formula (2): 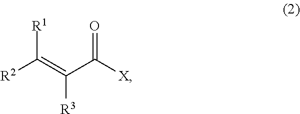
wherein R¹, R², R³, and X in the formula (2) are the same as those contained in the compound of formula (1).

4. The method according to claim 1, wherein the compound of formula (1) is at least one selected from the group consisting of propionic acid, butyric acid, pentanoic acid, hexanoic acid, isobutyric acid, 2-methylbutyric acid, 2-methylpentanoic acid, 2-methylhexanoic acid, and a methyl ester thereof.

5. The method according to claim 3, wherein the compound of formula (1) is isobutyric acid and the compound of formula (2) is methacrylic acid.

6. The method according to claim 3, wherein the compound of formula (1) is methyl isobutyrate and the compound of formula (2) is methyl methacrylate.

7. The method according to claim 1, wherein the photocatalyst comprises at least one selected from the group consisting of an inorganic photocatalyst and an organic photocatalyst.

8. The method according to claim 1, wherein the photocatalyst comprises at least one inorganic photocatalyst selected from the group consisting of a tungstic acid and a salt of a tungstic acid.

9. The method according to claim 1, wherein the photocatalyst comprises at least one inorganic photocatalyst selected from the group consisting of tetrabutylammonium decatungstate, sodium decatungstate, calcium tungstate, potassium tungstate, lithium tungstate, sodium tungstate, ammonium tungstate, cadmium tungstate, ammonium paratungstate, sodium polytungstate, (Bu₄N)₃PW₁₂O₄₀, (NH₄)₆P₂W₁₈O₆₂, (Bu₄N)₂W₆O₁₉, FeCl₃·6H₂O, FeCl₂·4H₂O, CuCl₂, and (TEA)₂CeCl₆.

10. The method according to claim 1, wherein the photocatalyst comprises a decatungstate.

11. The method according to claim 1, wherein the photocatalyst comprises tetrabutylammonium decatungstate.

12. The method according to claim 1, wherein the photocatalyst comprises at least one organic photocatalyst selected from the group consisting of benzophenone, bis(4-(trifluoromethyl)phenyl)methanone, bis(4-chlorophenyl)methanone, (4-methoxyphenyl) (4-(trifluoromethyl)phenyl)methanone, acetophenone, 9H-fluoren-9-one, phenanthrene-9,10-dione, pentacene-5,7,12,14-tetraone, 2-oxo-2-phenylacetic acid, 3a,5a-dihydropyrene-1,6-dione, anthracene-9,10-dione, 2-chloroanthraquinone, xanthone, and thioxanthone.

13. The method according to claim 1, wherein an amount of the photocatalyst is 0.5 mM or more and less than 4 mM.

14. The method according to claim 2, wherein the photocatalytic reaction occurs with irradiation from a light of a wavelength ranging from 300 nm to 425 nm and an irradiance ranging from 10 mW/cm² to 400 mW/cm².

15. The method according to claim 2, wherein the photocatalytic reaction occurs at an oxygen pressure ranging from 0 psig to 20 psig.

16. The method according to claim 2, wherein the photocatalytic reaction occurs in the presence of a solvent.

17. The method according to claim 16, wherein the solvent comprises at least one of acetonitrile and acetone.
